(19) United States
(12) Patent Application Publication
(10) Pub. No.: US 2004/0228930 A1
(43) Pub. Date: Nov. 18, 2004

(54) Treatment for hemorrhagic shock

(76) Inventors: Timothy R. Billiar, Nevillewood, PA (US); Augustine M.K. Choi, Pittsburgh, PA (US); Carol A. McCloskey, Pittsburgh, PA (US); Leo E. Otterbein, New Kensington, PA (US); Brian Zuckerbraun, Pittsburgh, PA (US)

Correspondence Address: FISH & RICHARDSON PC, 225 FRANKLIN ST, BOSTON, MA, 02110, US

(21) Appl. No.: 10/676,280

(22) Filed: Sep. 30, 2003

Related U.S. Application Data
(60) Provisional application No. 60/424,804, filed on Nov. 7, 2002.

(51) Int. Cl. A61K 33/00

(52) U.S. Cl. 424/699

(57) ABSTRACT

The present invention relates to methods and compositions of treating patients suffering from, or at risk for, hemorrhagic shock. The treatment includes administering to the patient a pharmaceutical composition that includes carbon monoxide.

### CROSS-REFERENCE TO RELATED APPLICATIONS

This application claims priority to U.S. Provisional Application No. 60/424,804, filed Nov. 7, 2002, which is incorporated herein by reference in its entirety.

### STATEMENT AS TO FEDERALLY SPONSORED RESEARCH

This invention was made with Government support under National Institutes of Health Grant No. P50-GM-53789. The Government has certain rights in this invention.

### TECHNICAL FIELD

This invention relates to the treatment of patients suffering from hemorrhagic shock.

### BACKGROUND

Heme oxygenase-1 (HO-1) catalyzes the first step in the degradation of heme. HO-1 cleaves the α-meso carbon bridge of b-type heme molecules by oxidation to yield equimolar quantities of biliverdin IXa, carbon monoxide (CO), and free iron. Subsequently, biliverdin is converted to bilirubin via biliverdin reductase, and the free iron is sequestered into ferritin (the production of which is induced by the free iron).

CO is recognized as an important signaling molecule (Verma et al., Science 259:381-384, 1993). It has been suggested that carbon monoxide acts as a neuronal messenger molecule in the brain (Id.) and as a neuro-endocrine modulator in the hypothalamus (Pozzoli et al., Endocrinology 735:2314-2317, 1994). Like nitric oxide, CO is a smooth muscle relaxant (Utz et al., Biochem Pharmacol. 47:195-201, 1991; Christodoulides et al., Circulation 97:2306-9, 1995) and inhibits platelet aggregation (Mansouri et al., Thromb Haemost. 48:286-8, 1982). Inhalation of low levels of CO has been shown to have anti-inflammatory effects in some models.

Hemorrhagic shock (or “HS”) is shock brought on by a loss of circulating blood volume and/or oxygen carrying capacity. HS may result from any condition associated with blood loss, e.g., internal (e.g., gastrointestinal bleeding) or external hemorrhage, and trauma (e.g., penetrating or blunt trauma), among others.

### SUMMARY

The present invention features a method of HS in a patient. The method includes administering to a patient diagnosed as suffering from, or at risk for, HS, an amount of a carbon monoxide-containing composition effective to reduce HS, e.g., the systemic tissue damage resulting from the HS. The method can include administering another treatment to the patient, such as fluid resuscitation, rehydration, oxygenation, surgery (e.g., to stop bleeding in the patient), vasoactive agent therapy, and/or antibiotic therapy.

The invention also features a method of treating HS in a patient by: (a) identifying a patient suffering from, or at risk for, HS, (b) administering fluid resuscitation to the patient, and (c) prior to, simultaneously with, or following (b), administering to the patient a pharmaceutical composition that includes carbon monoxide, in an amount effective treat HS, e.g., to reduce tissue damage (e.g., tissue damage to at least one organ, or systemic tissue damage) resulting from the HS.

Fluid resuscitation generally includes administering a liquid to the patient, particularly by administering it directly to a blood vessel (e.g., intravenously or intraarterially). The liquid can be, e.g., a liquid carbon monoxide composition (e.g., carbon monoxide-saturated Ringer's Solution, with or without lactate). Further, fluid resuscitation can include administering blood to the patient. The blood can be whole and/or partial (e.g., packed red blood cells, platelets, plasma, and/or coagulation factor precipitates) blood (e.g., diluted with an aqueous solution such as Ringer's solution), and can be completely or partially saturated with carbon monoxide.

The pharmaceutical composition can be in liquid or gaseous form, and can be administered to the patient by any method known in the art for administering gases and/or liquids to patients, e.g., via inhalation, insufflation, infusion (e.g., intravenously), injection, and/or ingestion. Alternatively or in addition, the composition can be administered topically, e.g., topically to an organ of the patient other than the lungs. In one embodiment of the present invention, the pharmaceutical composition is administered to the patient by inhalation. In another embodiment, the pharmaceutical composition is administered to the patient orally. In still another embodiment, the pharmaceutical composition is administered directly to the abdominal cavity of the patient.

The invention also provides a method of treating or preventing hemorrhagic shock in a patient, which includes administering to a patient diagnosed as suffering from blood loss (e.g., substantial blood loss (e.g., a loss of greater than about 15% total blood volume, e.g., greater than 20%, 25%, 30%, 35%, 40%, or 50% total volume, or at least 1000 ml, e.g., at least 1500, or at least 2000 ml, or any amount sufficient to cause hemorrhagic shock in the patient) or a lowered systolic blood pressure (e.g., a systolic blood pressure that is about 20 mmHg lower than the patient's normal systolic blood pressure or, e.g., a systolic blood pressure of less than about 100 mmHg, e.g., less than about 90, 60, or 50 mmHg) whole blood, or a blood component, containing an amount of dissolved CO effective to reduce systemic tissue damage resulting from the hemorrhagic shock. In certain embodiments, the patient is undergoing or has undergone a medical procedure, e.g., surgery or child birth.

Also included in the present invention is a method of performing a transfusion in a patient. The method includes (a) providing whole blood or a blood component suitable for transfusion into a patient; (b) saturating the blood or blood component partially or completely with carbon monoxide; and (c) infusing the partially or completely saturated blood or blood component into the patient. In certain embodiments, the patient is diagnosed as suffering from or at risk for hemorrhagic shock.

The present invention also includes a method of treating hemorrhagic shock in a patient, which includes (a) identifying a patient suffering from or at risk for hemorrhagic shock; (b) providing a vessel containing a pressurized gas comprising carbon monoxide gas; (c) releasing the pressurized gas from the vessel, to form an atmosphere comprising carbon monoxide gas; and (d) exposing the patient to the atmosphere, wherein the amount of carbon monoxide in the atmosphere is sufficient to reduce systemic tissue damage resulting from the hemorrhagic shock. The patient can be exposed to the atmosphere, e.g., continuously for at least one hour, e.g., at least 6, 24, 48, or 72 hours, or more.

In certain embodiments, the methods for treating hemorrhagic shock described herein further include monitoring the patient for signs of hemorrhagic shock. In other embodiments, the methods include observing a reduced level of systemic tissue damage than would have occurred in the absence of effective treatment.

A vessel that includes medical grade compressed carbon monoxide gas is also included within the present invention. The vessel can bear a label indicating that the gas can be used to treat or prevent HS in a patient, e.g., deleterious seqeulae of HS, e.g., systemic inflammation and/or the systemic tissue injury resulting from HS. The CO gas can be supplied as an admixture with nitrogen gas, with nitric oxide and nitrogen gas, or with an oxygen-containing gas. The CO gas can be present in the admixture at a concentration of at least about 0.025%, e.g., at least about 0.05%, 0.10%, 0.50%, 1.0%, 2.0%, 10%, 50%, or 90%, or greater.

In another aspect, the invention includes whole blood, or a blood component, that is partially or completely saturated with carbon monoxide, e.g., for transfusion into a patient to treat or prevent HS in a patient. For example, the invention includes whole blood or a blood component in a vessel (such as a blood bag suitable for a transfusion procedure), wherein the whole blood or blood component is partially or completely saturated with CO. The vessel can bear a label indicating that the whole blood or blood component can be used to treat or prevent HS, e.g., the systemic tissue damage that can result from HS.

In still another aspect, the invention includes a business method that includes: (a) providing whole blood or a blood component suitable for transfusion into a patient; (b) treating the blood (e.g., whole blood or partial blood) with carbon monoxide (e.g., exposing the blood to an atmosphere comprising carbon monoxide) to produce a blood/carbon monoxide product; and (c) supplying the blood/carbon monoxide product to a customer (e.g., a hospital or caregiver) with instructions to administer the blood/carbon monoxide product to a patient in need of a transfusion (e.g., due to a significant loss of blood).

Also within the invention is the use of CO in the manufacture of a medicament for treatment or prevention of HS, e.g., the tissue damage (e.g., systemic tissue damage) resulting from HS. The medicament can be used in a method for treating HS and/or the tissue damage resulting from hemorrhagic shock, and/or in a method for transfusing blood into a patient. The medicament can be in any form described herein, e.g., a liquid or gaseous CO composition.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a bar graph that illustrates the effect of CO on serum IL-6 levels in mice subjected to HS/R. N=3-4/group.

FIG. 2 is a bar graph that illustrates the effect of CO on serum IL-10 levels in mice subjected to HS/R. N=3-4/group.

FIG. 3 is a bar graph that illustrates the effect of CO on serum alanine aminotransferase (ALT) levels in mice subjected to HS/R. N=3-4/group.

FIGS. 4A-D are photographs of intestinal sections that illustrate the effect of CO on intestinal injury in mice subjected to HS/R. 4A: Air exposed mouse not subjected to HS/R. 4B: Air exposed mouse subjected to HS/R. 4C: CO exposed mouse not subjected to HS/R. 4D: CO exposed mouse subjected to HS/R. N=3-4/group.

FIG. 5A is a bar graph that illustrates the effect of CO on myeloperoxidase (MPO) activity in the lungs of mice subjected to HS/R when CO is administered during fluid resuscitation only.

FIG. 5B is a bar graph that illustrates the effect of CO on serum ALT levels in mice subjected to HS/R when CO is administered during fluid resuscitation only. N=3-4/group.

FIG. 6 is a bar graph that illustrates the effect of CO on MPO activity in the lungs of mice subjected to HS/R.

FIG. 7 is a bar graph that illustrates the effect of CO on hemorrhage-induced liver hypoxia.

FIG. 8A is a bar graph that illustrates the effect of CO on serum ALT levels in il-10^{-/-} mice subjected to HS/R.

FIG. 8B is a bar graph that illustrates the effect of CO on MPO activity in the lungs of il-10^{-/-} mice subjected to HS/R.

### DETAILED DESCRIPTION

The present invention is based, in part, on the discovery that CO administration affects cytokine levels and the occurrence of organ injury in animals subjected to HS followed by fluid resuscitation (HS/R).

The term “carbon monoxide” (or “CO”) as used herein describes molecular carbon monoxide in its gaseous state, compressed into liquid form, or dissolved in aqueous solution. The terms “carbon monoxide composition” and “pharmaceutical composition comprising carbon monoxide” is used throughout the specification to describe a gaseous or liquid composition containing carbon monoxide that can be administered to a patient and/or an organ, e.g., an organ affected by HS. Skilled practitioners will recognize which form of the pharmaceutical composition, e.g., gaseous, liquid, or both gaseous and liquid forms, is preferred for a given application.

The terms “effective amount” and “effective to treat,” as used herein, refer to an amount or a concentration of carbon monoxide utilized for a period of time (including acute or chronic administration and periodic or continuous administration) that is effective within the context of its administration for causing an intended effect or physiological outcome. Effective amounts of carbon monoxide for use in the present invention include, for example, amounts that reduce injury to a specific organ(s) effected by HS, or generally improve the a patient's prognosis following HS. The term “treat(ment)” is used herein to describe delaying the onset of, inhibiting, or alleviating the detrimental effects of a condition, e.g., organ injury/failure associated with or caused by HS.

For gases, effective amounts of CO generally fall within the range of about 0.0000001% to about 0.3% by weight, e.g., about 0.0001% to about 0.25% by weight, preferably at least about 0.001%, e.g., at least about 0.005%, 0.010%, 0.02%, 0.025%, 0.03%, 0.04%, 0.05%, 0.06%, 0.08%, 0.10%, 0.15%, 0.20%, 0.22%, or 0.24% by weight of CO. Preferred ranges of CO include 0.002% to about 0.24%, about 0.005% to about 0.22%, about 0.01% to about 0.20%, and about 0.02% to about 0.1% by weight. For liquid solutions of CO, effective amounts generally fall within the range of about 0.0001 to about 0.0044 g CO/100 g liquid, e.g., at least about 0.0001, 0.0002, 0.0004, 0.0006, 0.0008, 0.0010, 0.0013, 0.0014, 0.0015, 0.0016, 0.0018, 0.0020, 0.0021, 0.0022, 0.0024, 0.0026, 0.0028, 0.0030, 0.0032, 0.0035, 0.0037, 0.0040, or 0.0042 g CO/100 g aqueous solution. Preferred ranges include, e.g., about 0.0010 to about 0.0030 g CO/100 g liquid, about 0.0015 to about 0.0026 g CO/100 g liquid, or about 0.0018 to about 0.0024 g CO/100 g liquid. A skilled practitioner will appreciate that amounts outside of these ranges may be used depending upon the application.

The term “patient” is used throughout the specification to describe an animal, human or non-human, rodent or non-rodent, to whom treatment according to the methods of the present invention is provided. Veterinary and non-veterinary applications are contemplated. The term includes but is not limited to birds, reptiles, amphibians, and mammals, e.g., humans, other primates, pigs, rodents such as mice and rats, rabbits, guinea pigs, hamsters, cows, horses, cats, dogs, sheep and goats. Preferred subjects are humans, farm animals, and domestic pets such as cats and dogs.

The term “organ(s)” is used throughout the specification as a general term to describe any anatomical part or member having a specific function in an animal. Further included within the meaning of this term are portions of organs. Such organs include but are not limited to kidney, liver, heart, intestine, e.g., large or small intestine, pancreas, spleen, brain, and lungs.

The term “hemorrhagic shock” or “HS” as used herein generally refers to shock brought on by a loss (e.g., an acute or chronic loss) of circulating blood volume and/or oxygen carrying capacity. Hemorrhagic shock followed by resuscitation (HS/R) causes a systemic inflammatory response and often leads to organ injury and failure. The injury occurring following hemorrhagic shock is unique in that there is a global insult to all organ systems. The inability to meet the cellular metabolic demands results in rapid tissue injury and organ dysfunction. Outward symptoms of HS include, e.g., reduced urine output (e.g., oliguria or anuria), delayed capillary refill, increased heart rate, cool and clammy skin, compromised mental status (e.g., confusion, agitation, or lethargy), weakness, and increased respiration rate. A skilled practitioner will appreciate that hemorrhagic shock can be caused by any factor or condition that results in a substantial loss of blood from a patient, e.g., trauma (e.g., penetrating or blunt trauma), surgery, childbirth, and internal/external hemorrhages. A standard treatment for hemorrhagic shock is fluid resuscitation.

Individuals considered at risk for HS may benefit particularly from the invention, primarily because prophylactic treatment can begin before there is any evidence of HS. Individuals “at risk” include, e.g., individuals suffering from any condition described above, or having another factor that may put a patient at risk for blood loss, e.g., a chronic or hereditary disorder (e.g., hemophilia). For example, a person suffering from a wound (e.g., blunt trauma, a stab wound, or surgery) or a gastrointestinal bleed that has not yet lost a volume of blood sufficient to cause HS, can be treated according to the methods of the present invention before HS occurs.

Skilled practitioners will appreciate that a patient can be determined to be at risk for HS by any method known in the art, e.g., by a physician's diagnosis. Skilled practitioners will also appreciate that carbon monoxide compositions need not be administered to a patient by the same individual who diagnosed the patient (or prescribed the carbon monoxide composition for the patient). Carbon monoxide compositions can be administered (and/or administration can be supervised), e.g., by the diagnosing and/or prescribing individual, and/or any other individual, including the patient her/himself (e.g., where the patient is capable of self-administration).

Amounts of CO effective to treat hemorrhagic shock can be administered to (or prescribed for) a patient, e.g., by a physician or veterinarian, on the day the patient is diagnosed as suffering hemorrhagic shock, or as having any risk factor associated with an increased likelihood that the patient will develop hemorrhagic shock (e.g., the patient has recently lost, is losing, or is expected to lose a substantial amount of blood, e.g., due to a wound). Patients can inhale CO at concentrations ranging from 10 ppm to 3000 ppm, e.g., about 100 ppm to about 800 ppm, about 150 ppm to about 600 ppm, or about 200 ppm to about 500 ppm. Preferred concentrations include, e.g., about 30 ppm, 50 ppm, 75 ppm, 100 ppm, 125 ppm, 200 ppm, 250 ppm, 500 ppm, 750 ppm, or about 1000 ppm. CO can be administered to the patient intermittently or continuously. CO can be administered for at least about 1, 2, 4, 6, 8, 10, 12, 14, 18, or 20 days, e.g., 1, 2, 3, 5, or 6 months, or until the patient no longer exhibits symptoms of the condition or disorder, or until the patient is diagnosed as no longer being at risk for HS or organ injury from the aftermath of HS. In a given day, CO can be administered continuously for the entire day, or intermittently, e.g., a single whiff of CO per day (where a high concentration is used), or for up to 23 hours per day, e.g., up to 20, 15, 12, 10, 6, 3, or 2 hours per day, or up to 1 hour per day.

With regard to medical procedures, e.g., surgery and/or childbirth, CO can be administered systemically or locally to a patient prior to, during, and/or after the procedure is performed. Patients can inhale CO at concentrations ranging from 10 ppm to 1000 ppm, e.g., about 100 ppm to about 800 ppm, about 150 ppm to about 600 ppm, or about 200 ppm to about 500 ppm. Preferred concentrations include, e.g., about 30 ppm, 50 ppm, 75 ppm, 100 ppm, 125 ppm, 200 ppm, 250 ppm, 500 ppm, 750 ppm, or about 1000 ppm. CO can be administered to the patient intermittently or continuously, for at least about 1 hour, 2 hours, 3 hours, 4 hours, 6 hours, 12 hours, or at least about about 1, 2, 4, 6, 8, 10, 12, 14, 18, or 20 days, before the procedure. It can be administered in the time period immediately prior to the procedure and optionally continue through the procedure, or the administration can cease just prior to the procedure or at least 15 minutes before the procedure begins (e.g., at least 30 minutes, 1 hour, 2 hours, 3 hours, 6 hours, or 24 hours before the surgery begins). Alternatively or in addition, CO can be administered to the patient during the procedure, e.g., by inhalation and/or topical administration. Alternatively or in addition, CO can be administered to the patient after the procedure, e.g., starting immediately after completion of the procedure, and continuing for at least about 1, 2, 3, 5, 7, or 10 hours, or at least about 1, 2, 5, 8, 10, 20, 30, 50, or 60 days, 1 year, indefinitely, or until the patient no longer suffers from, or is at risk for, HS or organ injury after the completion of the procedure.

Preparation of Gaseous Compositions

A CO composition may be a gaseous composition. Compressed or pressurized gas useful in the methods of the invention can be obtained from any commercial source, and in any type of vessel appropriate for storing compressed gas. For example, compressed or pressurized gases can be obtained from any source that supplies compressed gases, such as oxygen, for medical use. The term “medical grade” gas, as used herein, refers to gas suitable for administration to patients as defined herein. The pressurized gas including CO used in the methods of the present invention can be provided such that all gases of the desired final composition (e.g., CO, He, NO, CO₂, O₂, N₂) are in the same vessel, except that NO and O₂ cannot be stored together. Optionally, the methods of the present invention can be performed using multiple vessels containing individual gases. For example, a single vessel can be provided that contains carbon monoxide, with or without other gases, the contents of which can be optionally mixed with the contents of other vessels, e.g., vessels containing oxygen, nitrogen, carbon dioxide, compressed air, or any other suitable gas or mixtures thereof.

Gaseous compositions administered to a patient according to the present invention typically contain 0% to about 79% by weight nitrogen, about 21% to about 100% by weight oxygen and about 0.0000001% to about 0.3% by weight (corresponding to about 1 ppb or 0.001 ppm to about 3,000 ppm) CO. Preferably, the amount of nitrogen in the gaseous composition is about 79% by weight, the amount of oxygen is about 21% by weight and the amount of CO is about 0.0001% to about 0.25% by weight, preferably at least about 0.001%, e.g., at least about 0.005%, 0.01%, 0.02%, 0.025%, 0.03%, 0.04%, 0.05%, 0.06%, 0.08%, 0.10%, 0.15%, 0.20%, 0.22%, or 0.24% by weight. Preferred ranges of CO include 0.005% to about 0.24%, about 0.01% to about 0.22%, about 0.015% to about 0.20%, about 0.08% to about 0.20%, and about 0.025% to about 0.1% by weight. It is noted that gaseous CO compositions having concentrations of CO greater than 0.3% (such as 1% or greater) may be used for short periods (e.g., one or a few breaths), depending upon the application.

A gaseous CO composition may be used to create an atmosphere that comprises CO gas. An atmosphere that includes appropriate levels of CO gas can be created, for example, by providing a vessel containing a pressurized gas comprising CO gas, and releasing the pressurized gas from the vessel into a chamber or space to form an atmosphere that includes the CO gas inside the chamber or space. Alternatively, the gases can be released into an apparatus that culminates in a breathing mask or breathing tube, thereby creating an atmosphere comprising CO gas in the breathing mask or breathing tube, ensuring the patient is the only person in the room exposed to significant levels of CO.

CO levels in an atmosphere or a ventilation circuit can be measured or monitored using any method known in the art. Such methods include electrochemical detection, gas chromatography, radioisotope counting, infrared absorption, colorimetry, and electrochemical methods based on selective membranes (see, e.g., Sunderman et al., Clin. Chem. 28:2026-2032, 1982; Ingi et al., Neuron 16:835-842, 1996). Sub-parts per million CO levels can be detected by, e.g., gas chromatography and radioisotope counting. Further, it is known in the art that CO levels in the sub-ppm range can be measured in biological tissue by a midinfrared gas sensor (see, e.g., Morimoto et al., Am. J. Physiol. Heart. Circ. Physiol 280:H482-H488, 2001). CO sensors and gas detection devices are widely available from many commercial sources.

Preparation of Liquid Compositions

A pharmaceutical composition comprising CO may also be a liquid composition. A liquid can be made into a pharmaceutical composition comprising CO by any method known in the art for causing gases to become dissolved in liquids. For example, the liquid can be placed in a so-called “CO₂ incubator” and directly exposed to a continuous flow of CO until a desired concentration of CO is reached in the liquid. As another example, CO gas can be “bubbled” directly into the liquid until the desired concentration of CO in the liquid is reached. The amount of CO that can be dissolved in a given aqueous solution increases with decreasing temperature. As still another example, an appropriate liquid may be passed through tubing that allows gas diffusion, where the tubing runs through an atmosphere comprising CO (e.g., utilizing a device such as an extracorporeal membrane oxygenator), or alternatively the gas is pumped through the lumen of the tubing and the liquid surrounds and is in contact with the exterior of the tubing. Either way, the CO diffuses into the liquid to create a liquid CO composition.

It is likely that such a liquid composition intended to be introduced into a living animal will be at or about 37° C. at the time it is introduced into the animal.

The liquid can be any liquid known to those of skill in the art to be suitable for administration to patients (see, for example, Oxford Textbook of Surgery, Morris and Malt, Eds., Oxford University Press (1994)). In general, the liquid will be an aqueous solution. Examples of solutions include Phosphate Buffered Saline (PBS), Celsior™, Perfadex™, Collins solution, citrate solution, and University of Wisconsin (UW) solution (Oxford Textbook of Surgery, Morris and Malt, Eds., Oxford University Press (1994)). In one embodiment of the present invention, the liquid is Ringer's Solution, e.g., lactated Ringer's Solution, or any other liquid that can be used for fluid resuscitation. In another embodiment, the liquid includes blood, e.g., whole blood, one or more individual blood components, and/or artificial blood substitute. The blood can be completely or partially saturated with carbon monoxide.

Any suitable liquid can be saturated to a set concentration of CO via gas diffusers. Alternatively, pre-made solutions that have been quality controlled to contain set levels of CO can be used. Accurate control of dose can be achieved via measurements with a gas permeable, liquid impermeable membrane connected to a CO analyzer. Solutions can be saturated to desired effective concentrations and maintained at these levels.

Treatment of Patients with CO Compositions

A patient can be treated with a carbon monoxide composition using any method known in the art of administering gases and/or liquids to patients. Carbon monoxide compositions can be prescribed for and/or administered to a patient diagnosed with, or determined to be at risk for, e.g., HS. The present invention contemplates the systemic administration of liquid or gaseous carbon monoxide compositions to patients (e.g., by inhalation and/or ingestion), and the topical administration of the compositions to the patient's organs in situ (e.g., by ingestion, insufflation, and/or introduction into the abdominal cavity). The compositions can be administered and/or supervised by any person, e.g., a health-care professional, veterinarian, or caretaker (e.g., an animal (e.g., dog or cat) owner), depending upon the patient to be treated, and/or by the patient him/herself, if the patient is capable of doing so. The present invention contemplates that agents capable of delivering doses of gaseous CO compositions or liquid CO compositions (e.g., CO-releasing gums, creams, ointments, lozenges, patches, or bandages) can be employed in addition or alternative to the modes for administering CO to patients described below.

Systemic Delivery of Gaseous CO

Gaseous CO compositions can be delivered systemically to a patient, e.g., a patient diagnosed with or determined to be at risk for HS. Gaseous CO compositions are typically administered by inhalation through the mouth or nasal passages to the lungs, where the CO is readily absorbed into the patient's bloodstream. The concentration of active compound (CO) utilized in the therapeutic gaseous composition will depend on absorption, distribution, inactivation, and excretion (generally, through respiration) rates of the CO as well as other factors known to those of skill in the art. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. Treatments can be monitored and CO dosages can be adjusted to ensure optimal treatment of the patient. Acute, sub-acute and chronic administration of CO are contemplated by the present invention, depending upon, e.g., the severity of HS in the patient. CO can be delivered to the patient for a time (including indefinitely) sufficient to treat the condition and exert the intended pharmacological or biological effect.

The following are examples of some methods and devices that can be utilized to administer gaseous CO compositions to patients.

Ventilators

Medical grade CO (concentrations can vary) can be purchased mixed with air or another oxygen-containing gas in a standard tank of compressed gas (e.g., 21% O₂, 79% N₂). It is non-reactive, and the concentrations that are required for the methods of the present invention are well below the combustible range (10% in air). In a hospital setting, the gas presumably will be delivered to the bedside where it will be mixed with oxygen or house air in a blender to a desired concentration in ppm (parts per million), though it can also be supplied at a concentration that requires no further dilution with oxygen or air. The patient will inhale the gas mixture through a ventilator, which will be set to a flow rate based on patient comfort and needs. This is determined by pulmonary graphics (i.e., respiratory rate, tidal volumes etc.). Fail-safe mechanism(s) to prevent the patient from unnecessarily receiving greater than desired amounts of carbon monoxide can be designed into the delivery system. The patient's CO level can be monitored by studying (1) carboxyhemoglobin (COHb), which can be measured in venous blood, and (2) exhaled CO collected from a side port of the ventilator. CO exposure can be adjusted based upon the patient's health status and on the basis of the markers. If necessary, CO can be washed out of the patient by switching to 100% O₂ inhalation. CO is not metabolized; thus, whatever is inhaled will ultimately be exhaled except for a very small percentage that is converted to CO₂. CO can also be mixed with any level of O₂ to provide therapeutic delivery of CO without consequential hypoxic conditions.

Face Mask and Tent

A CO-containing gas mixture is prepared as above to allow passive inhalation by the patient using a facemask or tent. The concentration inhaled can be changed and can be washed out by simply switching over to 100% O₂— Monitoring of CO levels would occur at or near the mask or tent with a fail-safe mechanism that would prevent too high of a concentration of CO from being inhaled.

Portable Inhaler

Compressed CO can be packaged into a portable inhaler device and inhaled in a metered dose, for example, to permit intermittent treatment of a recipient who is not in a hospital setting. Different concentrations of CO could be packaged in the containers. The device could be as simple as a small tank (e.g., under 5 kg) of appropriately diluted CO with an on-off valve and a tube from which the patient takes a whiff of CO according to a standard regimen or as needed.

Intravenous Artificial Lung

An artificial lung (a catheter device for gas exchange in the blood) designed for O₂ delivery and CO₂ removal can be used for CO delivery. The catheter, when implanted, resides in one of the large veins and would be able to deliver CO at given concentrations either for systemic delivery or at a local site. The delivery can be a local delivery of a high concentration of CO for a short period of time at the site of an organ, e.g., in proximity to the liver (this high concentration would rapidly be diluted out in the bloodstream), or a relatively longer exposure to a lower concentration of CO (see, e.g., Hattler et al., Artif. Organs 18(11):806-812 (1994); and Golob et al., ASAIO J. 47(5):432-437 (2001)).

Normobaric Chamber

In certain instances, it would be desirable to expose the whole patient to CO. The patient would be inside an airtight chamber that would be flooded with CO at a level that does not endanger the patient, or at a level that poses an acceptable risk without the risk of bystanders being exposed. Upon completion of the exposure, the chamber could be flushed with air or another oxygen-containing gas lacking CO and samples could be analyzed by CO analyzers to ensure no CO remains before allowing the patient to exit the exposure system.

Systemic Delivery of Liquid CO Compositions

The present invention further contemplates that aqueous solutions comprising carbon monoxide can be created for systemic delivery to a patient, e.g., for oral delivery and/or by infusion into the patient, e.g., intravenously, intra-arterially, intraperitoneally, and/or subcutaneously. For example, liquid CO compositions, such as CO-saturated Ringer's Solution, can be infused into a patient during fluid resuscitation.

Alternatively or in addition, whole (or partial) blood partially or completely saturated with CO can be infused into the patient to treat or prevent HS. Skilled practitioners will appreciate that levels of CO present in blood can be monitored by studying the amount of carboxyhemoglobin (COHb) present in the blood. For example, blood that is partially saturated with CO can display a carboxyhemoglobin content of greater than 10%, e.g., greater than 15, 25, 30, 50, or 90%, or more, COHb. Whole or partial blood (e.g., plasma) can be partially or completely saturated with CO by any art-known method. Exemplary methods, but not the only known methods, for introducing gases such as CO into blood samples (e.g., donated blood) are described in U.S. Pat. No. 5,476,764, which is incorporated herein by reference in its entirety. Such methods could be used to prepare CO saturated (or partially saturated) blood for transfusion into a patient. Skilled practitioners will appreciate that CO could be introduced into the blood at a blood bank immediately after withdrawing the blood from a donor; or immediately prior to transfusing the blood into the patient (e.g., by emergency medical personnel at the site of an accident); or at a stage during storage or transport of the blood after donation and prior to transfusion.

Topical Delivery of CO

Alternatively or in addition, CO compositions can be applied directly to organs in patients suffering from, or at risk for, hemorrhagic shock. A gaseous composition can be directly applied to a patient's organs by any method known in the art for insufflating gases into a patient. In one known illustration of insufflation for other purposes, a gas, e.g., carbon dioxide, is insufflated into the gastrointestinal tract and the abdominal cavity of patients to facilitate examination during endoscopic and laproscopic procedures, respectively (see, e.g., Oxford Textbook of Surgery, Morris and Malt, Eds., Oxford University Press (1994)). Skilled practitioners will appreciate that similar procedures could be used to administer CO compositions directly to a patient's organs.

Liquid CO compositions can also be administered topically to a patient's organs. Liquid forms of the compositions can be administered by any method known in the art for administering liquids to patients. For example, the liquid composition can be administered orally, e.g., by causing the patient to ingest an encapsulated or unencapsulated dose of the liquid CO composition. As another example, liquids, e.g., saline solutions containing dissolved CO, can be injected into the gastrointestinal tract and/or the abdominal cavity of patients suffering from HS. Further, in situ exposures can be carried out by flushing an organ with a liquid CO composition (see Oxford Textbook of Surgery, Morris and Malt, Eds., Oxford University Press (1994)).

Use of Hemoxygenase-I and Other Compounds

Also contemplated by the present invention is the induction, expression, and/or administration of hemeoxygenase-1 (HO-1) in conjunction with administration of CO. HO-1 can be provided to a patient by inducing or expressing HO-1 in the patient, or by administering exogenous HO-1 directly to the patient. As used herein, the term “induce(d)” means to cause increased production of a protein, e.g., HO-1, in isolated cells or the cells of a tissue, organ or animal using the cells' own endogenous (e.g., non-recombinant) gene that encodes the protein.

HO-1 can be induced in a patient by any method known in the art. For example, production of HO-1 can be induced by hemin, by iron protoporphyrin, or by cobalt protoporphyrin. A variety of non-heme agents including heavy metals, cytokines, hormones, NO, COCl₂, endotoxin and heat shock are also strong inducers of HO-1 expression (Choi et al., Am. J. Respir. Cell Mol. Biol. 15:9-19, 1996; Maines, Annu. Rev. Pharmacol. Toxicol. 37:517-554, 1997; and Tenhunen et al., J. Lab. Clin. Med. 75:410-421, 1970). HO-1 is also highly induced by a variety of agents causing oxidative stress, including hydrogen peroxide, glutathione depletors, UV irradiation, endotoxin and hyperoxia (Choi et al., Am. J. Respir. Cell Mol. Biol. 15:9-19, 1996; Maines, Annu. Rev. Pharmacol. Toxicol. 37:517-554, 1997; and Keyse et al., Proc. Natl. Acad. Sci. USA 86:99-103, 1989). Alternatively or in addition, HO-1 protein can be directly administered to a patient, e.g., in liposomes. A “pharmaceutical composition comprising an inducer of HO-1” means a pharmaceutical composition containing any agent capable of inducing HO-1 in a patient, e.g., any of the agents described above, e.g., NO, hemin, iron protoporphyrin, and/or cobalt protoporphyrin.

HO-1 expression in a cell can be increased via gene transfer. As used herein, the term “express(ed)” means to cause increased production of a protein, e.g., HO-1 or ferritin, in isolated cells or the cells of a tissue, organ or animal using an exogenously administered gene (e.g., a recombinant gene). The HO-1 or ferritin is preferably of the same species (e.g., human, mouse, rat, etc.) as the patient, in order to minimize any immune reaction. Expression could be driven by a constitutive promoter (e.g., cytomegalovirus promoters) or a tissue-specific promoter (e.g., milk whey promoter for mammary cells or albumin promoter for liver cells). An appropriate gene therapy vector (e.g., retroviruses, adenoviruses, adeno associated viruses (AAV), pox (e.g., vaccinia) viruses, human immunodeficiency virus (HIV), the minute virus of mice, hepatitis B virus, influenza virus, Herpes Simplex Virus-1, and lentiviruses) encoding HO-1 or ferritin would be administered to the patient orally, by inhalation, or by injection at a location appropriate for treatment of a disorder or condition described herein. Particularly preferred is local administration directly to the affected site before, during, and/or after the development of HS. Similarly, plasmid vectors encoding HO-1 or apoferritin can be administered, e.g., as naked DNA, in liposomes, or in microparticles.

Further, exogenous HO-1 protein can be directly administered to a patient by any method known in the art. Exogenous HO-1 can be directly administered in addition to, or as an alternative, to the induction or expression of HO-1 in the patient as described above. The HO-1 protein can be delivered to a patient, for example, in liposomes, and/or as a fusion protein, e.g., as a TAT-fusion protein (see, e.g., Becker-Hapak et al., Methods 24: 247-256 (2001)).

Alternatively or in addition, any of the products of metabolism by HO-1, e.g., bilirubin, biliverdin, iron, and/or ferritin, can be administered to a patient in conjunction with CO to a patient suffering from, or at risk for, HS. Further, the present invention contemplates that iron-binding molecules other than ferritin, e.g., desferoxamine (DFO), iron dextran, and/or apoferritin, can be administered to the patient. Further still, the present invention contemplates that enzymes (e.g., biliverdin reductase) that catalyze the breakdown any of these products can be inhibited to create/enhance the desired effect. Any of the above can be administered, e.g., orally, intravenously, intraperitoneally, or topically.

The present invention contemplates that compounds that release CO into the body after administration of the compound (e.g., CO-releasing compounds, photoactivatable CO-releasing compounds) e.g., metal carbonyl compounds, dimanganese decacarbonyl, tricarbonyldichlororuthenium (II) dimer, and methylene chloride (e.g., at a dose of between 400 to 600 mg/kg, e.g., about 500 mg/kg) can also be used in the methods of the present invention, as can carboxyhemoglobin and CO-donating hemoglobin substitutes.

The above can be administered to a patient in any way, e.g., oral, intravenous, intraperitoneal, or intraarterial administration. Any of the above compounds can be administered to the patient locally and/or systemically, and in any combination.

Combination Therapy

Also contemplated by the present invention is administration of CO to a patient in conjunction with at least one other treatment for preventing/treating hemorrhagic shock. Such treatments include, e.g., measures to control bleeding (e.g., compression of external bleeding sites) and surgery (e.g., to stop bleeding in the patient). Whole blood transfusions can also be performed, as can transfusion of partial blood (i.e., one or more individual blood component (e.g., packed red blood cells, platelets, plasma, and/or coagulation factor precipitates), and mixtures of blood (or individual blood component(s)) with another liquid (e.g., diluted whole blood or individual blood component(s)). Also useful in the treatment or prevention of HS is administration of oral and/or intravenous rehydration, liquid resuscitation (e.g., using crystalloid, colloid, or blood products), oxygenation, vasoactive agent therapy (e.g., administration of inotropic (e.g., dopamine and dobutamine) and/or vasorepressor (e.g., phenylephrine, noroepinephrine, and epinephrine) agents), and antibiotic (e.g., broad spectrum antibiotic) therapy, among others.

The invention is illustrated in part by the following example, which is not to be taken as limiting the invention in any way.

### EXAMPLE 1

### Administration of CO Protects Organs in Animals Subjected to HS/R

The studies described below demonstrate that CO can protect against organ injury in a model of HS/R. In a mouse model of hemorrhagic shock-induced mutli-organ failure, exposure to a low concentration of CO imparted a potent defense against the inflammatory sequelae and end-organ damage that ensue following hemorrhage and resuscitation. CO effectively suppressed shock-induced lung, liver, and intestinal injury as determined by decreases in myeloperoxidase activity, serum alanine aminotransferase levels, and intestinal architectural changes, respectively. Additionally, CO paradoxically abrogated hemorrhage-induced hepatic cellular hypoxia. Taken together, these results demonstrate a protective role for CO in hemorrhagic shock-induced organ injury.

Because HS is a systemic injury, CO as a therapeutic agent has several potential benefits. For example, CO is capable of reaching all tissues and, therefore, of diminishing the progression of injury within each organ while decreasing activation of circulating inflammatory cells. As another example, CO can easily be administered, e.g., as an inhalational agent, in the field by emergency medical personnel (e.g., by mask and/or endotracheal tube).

Animals and Hemorrhagic Shock

Hemorrhagic shock was induced in mice as follows: C57/BL6 or il-10^{-/-} mice (Jackson Laboratories) (n=3/group) weighing 20-26 grams were anesthetized with pentobarbital (70 mg/kg; IP). The right and left femoral arteries were cannulated. The left arterial catheter was connected to a monitor to follow MAP and heart rate. Over 10 minutes, blood was withdrawn via the right catheter while monitoring blood pressure to achieve an MAP of 25 mmHg. Blood was withdrawn and returned to the animal as necessary in order to maintain a MAP of 25 mmHg. Sham animals were cannulated but not subjected to hemorrhage. At the end of the shock period (90-150 minutes), mice were resuscitated with the shed blood plus two times that volume with Ringer's lactate solution over 15-30 minutes. Animals were sacrificed 4-24 hours following the initiation of resuscitation and blood, liver, lungs, and intestines were collected.

Cytokine and Serum ALT Measurement

Serum levels of the cytokines interleukin-6 (IL-6), tumor necrosis factor alpha (TNF-α), and interleukin-10 (IL-10) were determined by enzyme-linked immunosorbent assay (ELISA) (R&D systems) according to the manufacturer's instructions. These cytokines were measured because they mediate inflammation. Levels of pro-inflammatory cytokines can increase following hemorrhagic shock and can exacerbate hemodynamics and contribute to the development of multiple organ dysfunction.

A colorimetric analysis for serum alanine aminotransferase (ALT) level was used to determine whether liver injury occurred in mice subjected to HS/R. The alanine aminotransferase test is one of a group of tests known as liver function tests and is used to monitor damage to the liver. Alanine aminotransferase is an enzyme that is normally expressed in the liver and under normal circumstances is present in serum samples at very low levels. Increased levels of ALT in the serum are indicative of liver cell death and/or liver failure.

Myeloperoxidase Activity

MPO activity in the lungs was observed as follows: lungs were excised, washed in saline, and frozen in liquid nitrogen. Samples were thawed and homogenized in 20 mmol/L potassium phosphate (pH 7.4). Samples were centrifuged at 15,000×g for 30 minutes at 4° C. to form a pellet. The pellet was resuspended in 50 mmol/L potassium phosphate (pH 6.0) containing 0.5% hesadecyltrimethylammonium bromide. Samples were sonicated and then centrifuged at 15,000×g for 10 minutes at 4° C. Five microliters of supernatant was then added to 196 μL of reaction buffer containing 530 nmol/L o-dianisidine and 150 nmol/L H₂O₂ in 50 mmol/L potassium phosphate (pH 6.0). Light absorbances (490 and 620 nm) were read and compared to standards. Protein content in the samples was determined using a bicinchoninic assay (BCA). Results were normalized to total amount of protein present.

Hypoxic Imaging

The technique of observing uptake of EF5 (2-[2-nitro-1H-imidazol-1-yl]-N-(2,2,3,3,3-pentafluoropropyl) acetamide) to monitor tissue hypoxia is well established and reliable. EF5 delivery and staining was performed as follows: EF5 (10 μl/g of a 10 mM stock; ip; Hypoxia Imaging Group, University of Pennsylvania) was provided to each animal 30 minutes after the onset of shock. Animals were sacrificed 90 minutes after the initiation of shock and livers were harvested for immunohistochemical analysis of EF5 according to the manufacturer's protocol. Intensity of staining was determined by measuring mean fluorescence in 10 different sections per animal (n=5 per group; MetaMorph®). EF5 is a nitroimidazole that gets taken up by all cells and can be reduced. Under normoxic conditions the electron from the reduced form of EF5 is transferred to oxygen and there is a ‘futile cycling’ of electrons. Under hypoxic conditions the nitroimidazole is further reduced to form nitroso- and hydroxylamines. These forms of the compound bind irreversibly to proteins within the cell, which can then be detected by immunohistochemistry. Thus, the extent of EF5 binding can be used as an indirect measure of oxygen tension. In the in vivo setting, positive EF5 staining is directly related to tissue hypoxia.

Histology

Harvested intestines were flushed and fixed in 2% paraformaldehyde for 2 hours and then 30% sucrose for 12 hours. Specimens were frozen slowly in cold 2-methylbutane. Sections were stained using hematoxylin and eosin (H&E) and architectural changes were evaluated.

CO Exposure

Mice were exposed to CO at a concentration of 250 ppm. Briefly, 1% CO in air was mixed with air (21% oxygen) in a stainless steel mixing cylinder and then directed into a 3.70 ft³ glass exposure chamber at a flow rate of 12 L/min. A CO analyzer (Interscan, Chatsworth, Calif.) was used to measure CO levels continuously in the chamber. CO concentrations were maintained at 250 ppm at all times. Mice were placed in the exposure chamber as required.

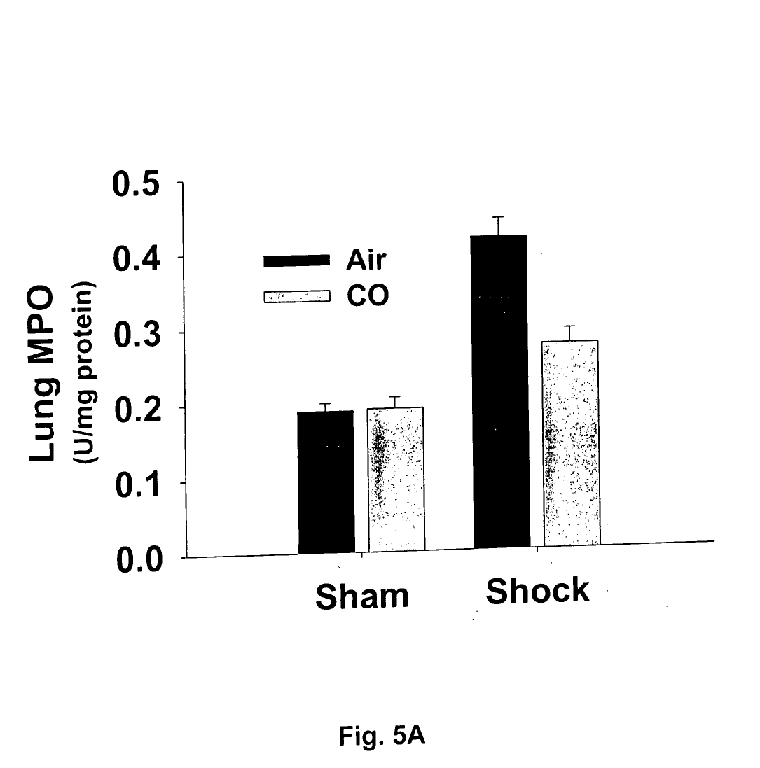

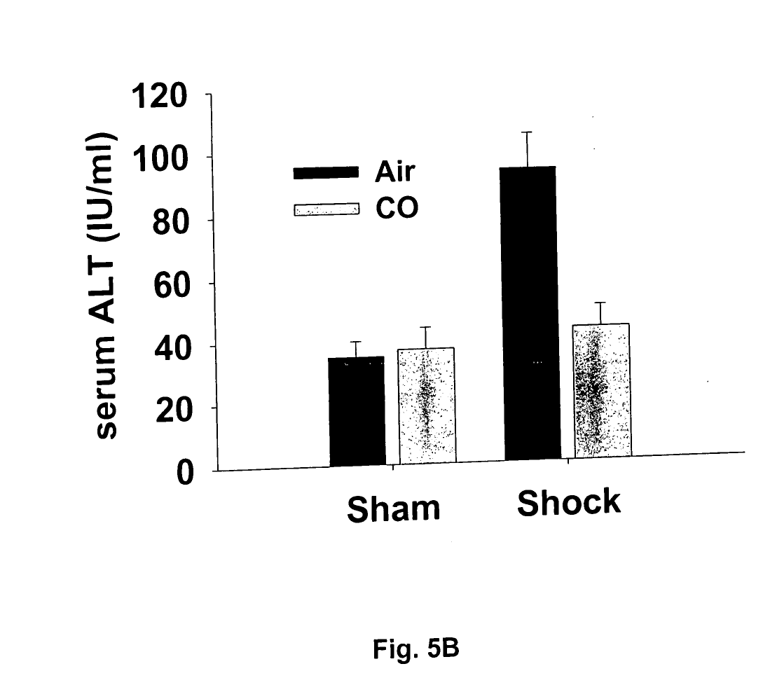

In most experiments, mice were treated with CO (250 ppm) or standard room air (control) throughout the duration of HS/R, i.e., CO administration commenced at the beginning of the 2.5 hour HS period and ended after the 4 hour fluid resuscitation period. However, in one experiment (where fluid resuscitation was performed for 24 hours), mice were treated with CO or room air during the resuscitation period only (see FIG. 5). In all cases, the mice were sacrificed following the resuscitation period.

Carbon Monoxide Protects Against Multiple Organ Injury in a Model of Hemorrhagic Shock and Resuscitation

Inhaled Carbon Monoxide does not Influence Central Hemodynamics

Both sham and shocked animals were anesthetized and had arterial and venous catheters inserted as discussed above. Mean arterial pressure (MAP) was monitored throughout the duration of ‘shock’ in both the sham and shock groups. CO treatment (250 ppm) did not alter either the MAP or the heart rate in the sham operated mice compared to air controls. Likewise, the volume of hemorrhaged blood that was shed in order to achieve a MAP of 25 mmHg was the same in both the CO-treated and air-treated shocked mice. A MAP of 25 mmHg is art-recognized as being a level at which hemorrhagic shock is produced in mice. Although CO is known to activate soluble guanylate cyclase and may possess vasodilator properties, there was no measurable effect on systemic blood pressure at the dose utilized in these studies. Table 1 (below) illustrates that CO administration does not affect MAP in healthy mice. Further, Table 1 shows that CO administration did not affect the volume of blood required to be removed from mice in order to achieve a MAP of 25 mmHg.

TABLE 1

| | MAP (Sham) | Shed Blood (Shock) |
|---|---|---|
| | mmHg | mL |
| Air | 67.2 ± 5.1 | 0.72 ± 08 |
| CO | 68.9 ± 6.0 | 0.69 ± 0.6 |

CO Decreases HSIR-Induced Serum IL-6 and Increases HSIR-Induced Serum IL-10 Levels

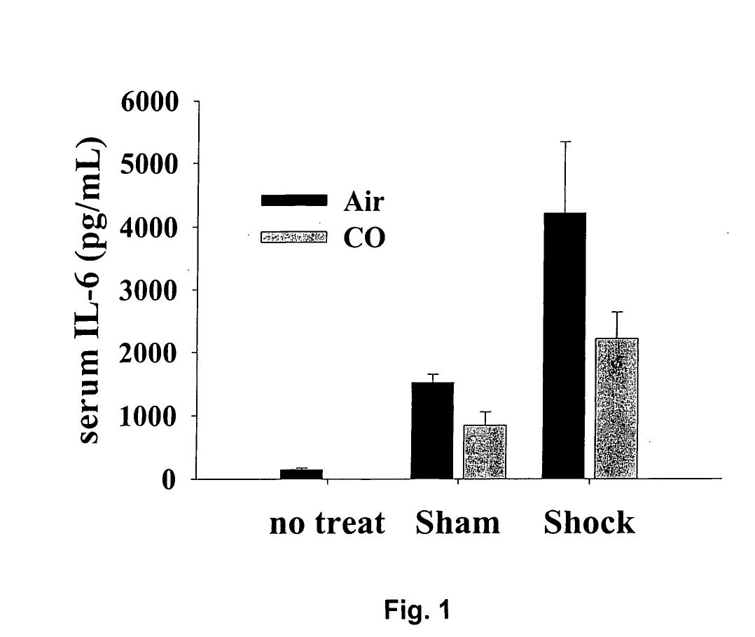

Levels of pro-inflammatory cytokines such as IL-6 can increase following hemorrhagic shock. These cytokines can exacerbate hemodynamics and contribute to the development of multiple organ dysfunction. Accordingly, the effects of CO on HS/R-induced increases in serum IL-6 levels were examined. Cytokine levels were examined 4 hours after resuscitation. Serum IL-6 levels in the HS/R group were 2.82-fold higher than sham controls (FIG. 1; P<0.05). This increase in IL-6 was significantly abrogated in those animals treated with CO(P<0.05 compared to the untreated HS/R mice). Thus, CO administration results in decreased serum IL-6 levels in animals subjected to HS/R. CO-induced decreases in IL-6 may be one mechanism by which CO confers protection

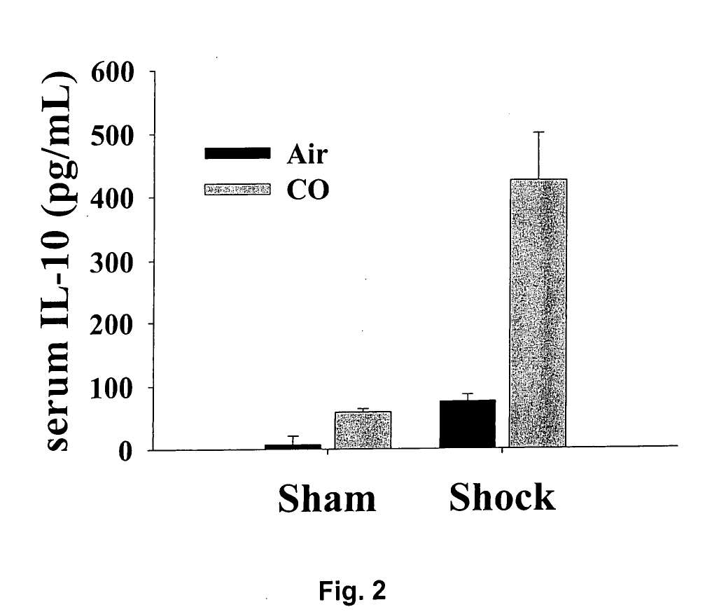

Additionally, the effects of CO on serum IL-10 (an anti-inflammatory cytokine) levels were examined. In this model of HS/R, CO treatment increased serum IL-10 levels in shocked mice by 5.4 fold (FIG. 2; P<0.05 compared to sham and shock controls). Thus, CO administration resulted in increased serum IL-10 levels in animals subjected to HS/R. CO-induced increases in levels of this anti-inflammatory cytokine may be another mechanism by which CO confers protection.

CO Decreases Liver, Lung, and Intestinal Injury Following HS/R.

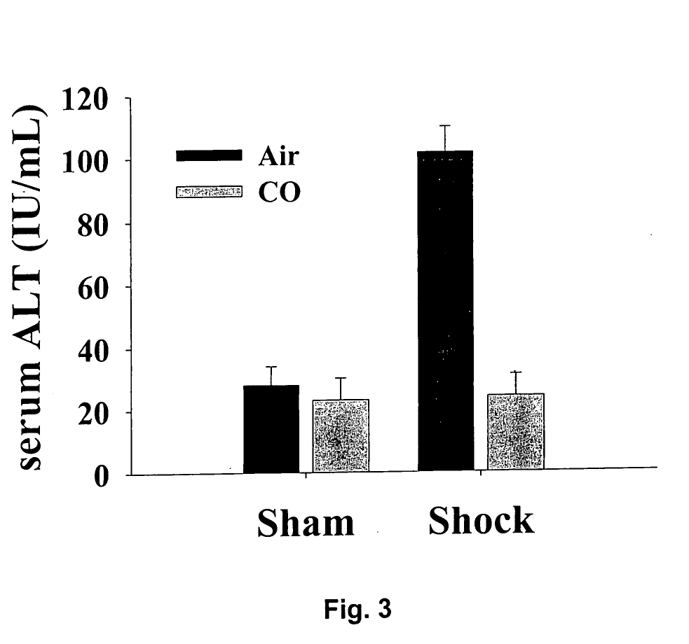

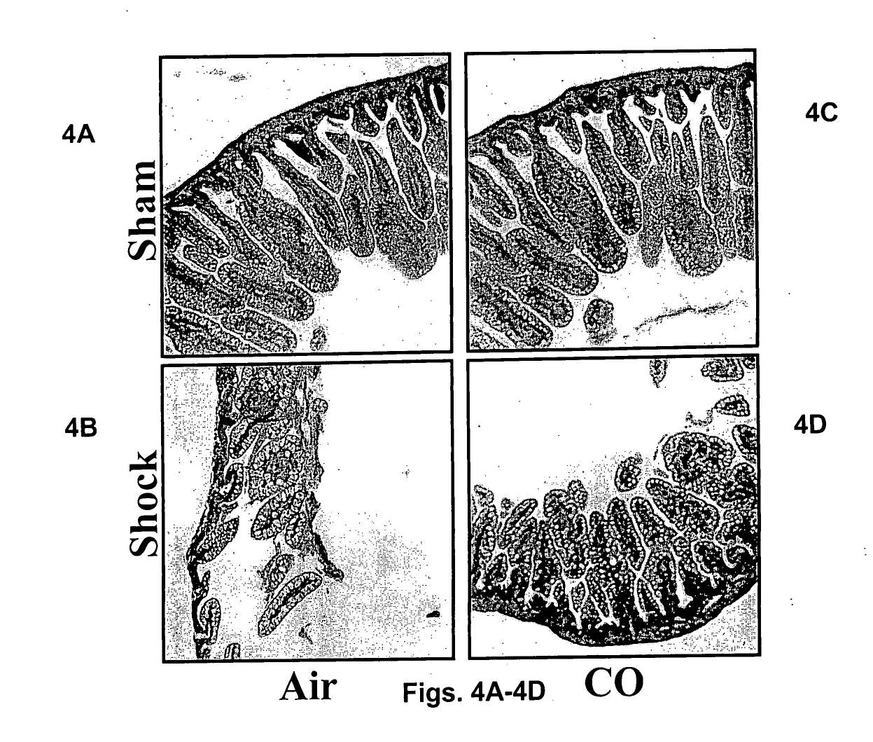

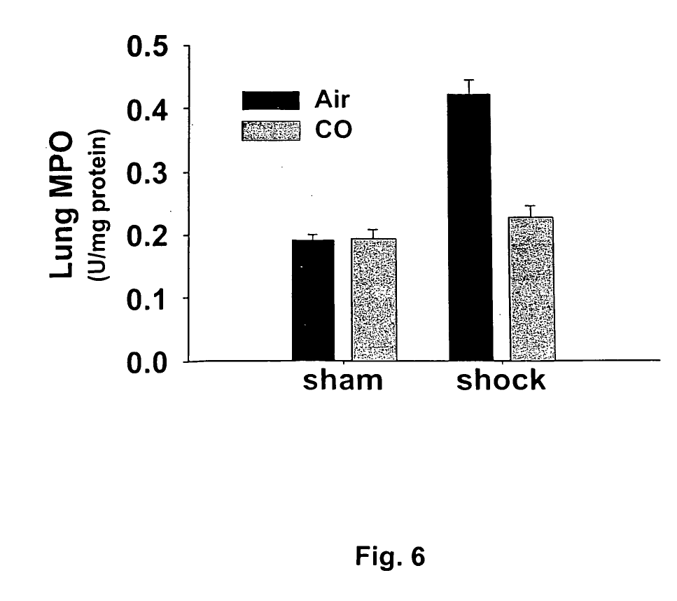

Whether CO could protect against organ injury induced by HS/R was investigated. Serum, liver, lung, and intestines were harvested 4 hours after resuscitation as discussed above. Liver, lung and intestinal injury were examined by studying serum ALT (FIG. 3), lung MPO activity (FIG. 6), and intestinal histology (FIGS. 4A to 4D), respectively. HS/R resulted in injury and tissue damage in all cases (see FIGS. 3, 4B, and 6). CO treatment, which had no measurable effect in sham animals, protected against these injuries. In those animals that received HS/R, CO significantly lowered serum ALT (FIG. 3) and lung MPO activity (FIG. 6) compared to untreated mice (P<0.05). With regard to intestinal injury, CO-treated shocked mice (FIG. 4D) had intestinal histology that more closely resembled CO- and air-treated sham controls (FIGS. 4C and 4A, respectively). Thus, CO administration appears to reduce liver, lung, and intestinal injury in animals subjected to HS/R.

Therapeutic CO Can Protect Against Organ Injury.

CO treatment in all of the experiments described above was initiated concurrently with hemorrhaging of the animals. Accordingly, whether delivery of CO initiated during the resuscitation period protects against organ injury was investigated. Mice were subjected to 2.5 hours of HS followed by 24 hours of fluid resuscitation. CO was administered to the mice during the 24 hour fluid resuscitation period (and not during the HS period). Although initiation of CO treatment during resuscitation significantly improved lung MPO activity after 4 hours (FIG. 5A), there was no demonstrable protection against liver injury when assayed at the 4-hour time point following resuscitation (data not shown). However, when liver injury was assayed at a later time point (24 hours after resuscitation), CO-treated mice had significantly lower levels of serum ALT compared to that of untreated shocked mice (FIG. 5B). Thus, CO administration appears to substantially reduce liver injury/failure in animals subjected to HS/R, even when CO treatment is delayed until commencement of fluid resuscitation.

Carbon Monoxide Decreases Liver Hypoxia

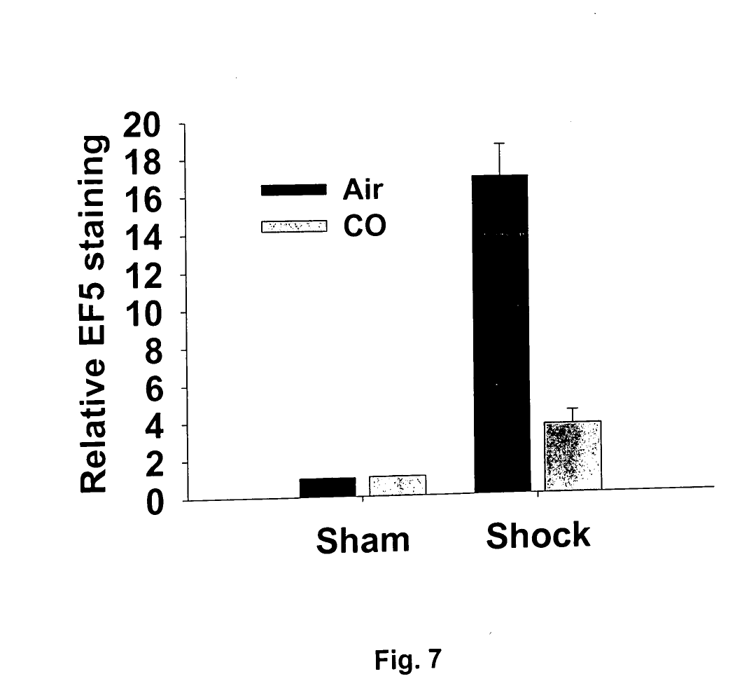

One mechanism by which CO may confer protection is by decreasing hemorrhage-induced tissue hypoxia. The effects of CO on tissue hypoxia were examined by utilizing the nitroimidazole EF5. Under hypoxic conditions EF5 is reduced and binds irreversibly to intracellular proteins. Samples can be immunostained against this compound to monitor cellular hypoxia. Sham and shocked mice were left untreated or treated with CO (250 ppm; initiated at the onset of shock). EF5 (10 μl/g of a 10 mM stock; ip) was administered to each animal 30 minutes after the onset of shock. Animals were sacrificed 90 minutes after the initiation of shock and livers were harvested for immunohistochemical analysis of EF5 as discussed above. There was a 17±1.7 fold increase in EF5 staining in the livers of air-treated shocked mice compared to air-treated sham controls (P<0.01, FIG. 7). EF5 staining was most notably increased around the central veins. CO treatment decreased EF5 staining in the livers of shocked animals, resulting in only a 3.7±0.7 fold increase in staining compared to air-treated sham controls (P<0.05 compared to air-treated shocked mice). CO-treated sham controls exhibited no increase in liver hypoxia compared to air-treated sham controls. These data suggest that CO decreases tissue hypoxia that occurs with hemorrhage.

CO does not Protect Against Organ Injury in il-10^{-/-} Mice

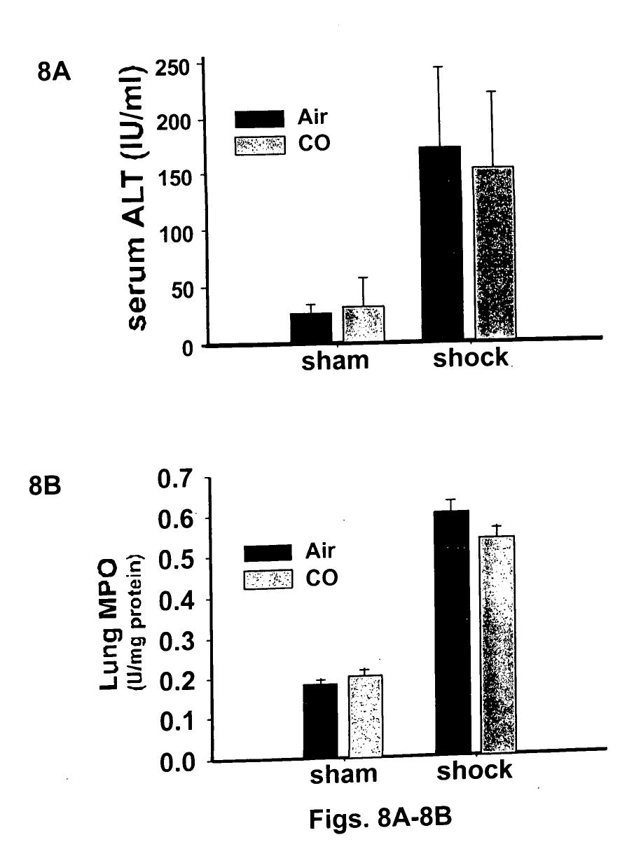

To determine whether protection is, in part, related to the ability of CO to increase IL-10 expression, HS/R was performed with and without administration of CO in IL-10-deficient mice (il-10^{-/-}). Using lung MPO activity and serum ALT as measurements of lung and liver injury, respectively, CO did not appear to protect against HS/R-induced organ injury in those mice (see FIGS. 8A and 8B). These results are consistent with the hypothesis that IL-10 may mediate some of the protective effects of CO. However, HS/R-induced injury in these il-10^{-/-} mice was more pronounced compared to their genetically matched wild-types (C57/BL6), as exemplified by greater increases in ALT and MPO following HS/R. The increased susceptibility of il-10^{-/-} mice to injury and exaggerated response to hemorrhage may account for the inability of CO to protect in these mice.

A number of embodiments of the invention have been described. Nevertheless, it will be understood that various modifications may be made without departing from the spirit and scope of the invention. Accordingly, other embodiments are within the scope of the following claims.

What is claimed is:

1. A method of treating hemorrhagic shock in a patient, comprising:
administering to a patient diagnosed as suffering from hemorrhagic shock an amount of a pharmaceutical composition comprising carbon monoxide effective to reduce tissue damage resulting from the hemorrhagic shock.

2. The method of claim 1, further comprising administering to the patient at least one treatment selected from the group consisting of: blood transfusion, rehydration, surgery, antibiotic therapy, and vasoactive drug therapy.

3. The method of claim 1, wherein the pharmaceutical composition is in gaseous form and is administered to the patient via inhalation.

4. The method of claim 1, wherein the pharmaceutical composition in is gaseous form and is administered topically to an organ of the patient other than the lung.

5. The method of claim 1, wherein the pharmaceutical composition in is gaseous form and is administered to the abdominal cavity of the patient.

6. The method of claim 1, wherein the pharmaceutical composition is in liquid form and is administered to the patient orally.

7. The method of claim 1, wherein the pharmaceutical composition is in liquid form and is administered topically to an organ of the patient.

8. The method of claim 1, wherein the pharmaceutical composition is in liquid form and is administered to the abdominal cavity of the patient.

9. The method of claim 1, wherein the pharmaceutical composition is in liquid form and is administered to the patient intravenously or intraperitoneally.

10. The method of claim 1, further comprising observing a reduced level of systemic tissue damage than would have occurred in the absence of effective treatment.

11. The method of claim 1, further comprising monitoring the patient for signs of hemorrhagic shock.

12. A method of treating hemorrhagic shock in a patient, comprising:
administering to a patient diagnosed as at risk for hemorrhagic shock an amount of a pharmaceutical composition comprising carbon monoxide effective to reduce systemic tissue damage resulting from the hemorrhagic shock; and
monitoring the patient for signs of hemorrhagic shock.

13. The method of claim 12, further comprising administering to the patient at least one treatment selected from the group consisting of: blood transfusion, rehydration, surgery, antibiotic therapy, and vasoactive drug therapy.

14. The method of claim 12, wherein the pharmaceutical composition is in gaseous form and is administered to the patient via inhalation.

15. The method of claim 12, wherein the pharmaceutical composition in is gaseous form and is administered topically to an organ of the patient other than the lung.

16. The method of claim 12, wherein the pharmaceutical composition in is gaseous form and is administered to the abdominal cavity of the patient.

17. The method of claim 12, wherein the pharmaceutical composition is in liquid form and is administered to the patient orally.

18. The method of claim 12, wherein the pharmaceutical composition is in liquid form and is administered topically to an organ of the patient.

19. The method of claim 12, wherein the pharmaceutical composition is in liquid form and is administered to the abdominal cavity of the patient.

20. The method of claim 12, wherein the pharmaceutical composition is in liquid form and is administered to the patient intravenously or intraperitoneally.

21. A method of treating hemorrhagic shock in a patient, comprising:
(a) identifying a patient suffering from, or at risk for, hemorrhagic shock;
(b) administering fluid resuscitation to the patient; and
(c) simultaneously with or following step (b), administering to the patient a pharmaceutical composition comprising carbon monoxide in an amount effective to reduce systemic tissue damage resulting from the hemorrhagic shock.

22. The method of claim 21, wherein administering fluid resuscitation comprises administering a liquid carbon monoxide composition to the patient.

23. The method of claim 21, wherein the liquid carbon monoxide composition is carbon monoxide-saturated Ringer's Solution.

24. The method of claim 21, wherein administering fluid resuscitation comprises administering to the patient blood that is partially or completely saturated with carbon monoxide.

25. The method of claim 21, wherein administering fluid resuscitation further comprises administering carbon monoxide-saturated Ringer's Solution to the patient.

26. The method of claim 21, wherein the pharmaceutical composition is in gaseous form and is administered to the patient via inhalation.

27. The method of claim 21, wherein the pharmaceutical composition in is gaseous form and is administered topically to an organ of the patient other than the lung.

28. The method of claim 21, wherein the pharmaceutical composition in is gaseous form and is administered to the abdominal cavity of the patient.

29. The method of claim 21, wherein the pharmaceutical composition is in liquid form and is administered to the patient orally.

30. The method of claim 21, wherein the pharmaceutical composition is in liquid form and is administered topically to an organ of the patient.

31. The method of claim 21, wherein the pharmaceutical composition is in liquid form and is administered to the abdominal cavity of the patient.

32. A method of treating hemorrhagic shock in a patient, comprising:
administering, to a patient diagnosed as suffering from blood loss possibly sufficient to cause hemorrhagic shock, whole blood, or a blood component, containing an amount of dissolved carbon monoxide effective to reduce systemic tissue damage resulting from the hemorrhagic shock.

33. The method of claim 32, wherein the patient is undergoing or has undergone surgery.

34. A method of performing a transfusion in a patient, comprising:
(a) providing whole blood or a blood component suitable for transfusion into a patient;
(b) saturating the whole blood or blood component partially or completely with carbon monoxide; and
(c) infusing the partially or completely saturated whole blood or blood component into the patient, to thereby perform a transfusion in a patient.

35. The method of claim 34, wherein the patient is diagnosed as suffering from or at risk for hemorrhagic shock.

36. A method of treating hemorrhagic shock in a patient, comprising:
(a) identifying a patient suffering from or at risk for hemorrhagic shock;
(b) providing a vessel containing a pressurized gas comprising carbon monoxide gas;
(c) releasing the pressurized gas from the vessel, to form an atmosphere comprising carbon monoxide gas; and
(d) exposing the patient to the atmosphere, wherein the amount of carbon monoxide in the atmosphere is sufficient to reduce systemic tissue damage resulting from the hemorrhagic shock.

37. The method of claim 36, wherein the patient is exposed to the atmosphere continuously for at least one hour.

38. The method of claim 36, wherein the patient is exposed to the atmosphere continuously for at least six hours.

39. The method of claim 36, wherein the patient is exposed to the atmosphere continuously for at least 24 hours.

40. The method of claim 36, further comprising monitoring a symptom of hemorrhagic shock in the patient.

41. A vessel comprising medical grade compressed carbon monoxide gas, the vessel bearing a label indicating that the gas can be used to reduce deleterious sequelae of hemorrhagic shock in a patient.

42. The vessel of claim 41, wherein the deleterious sequelae comprise systemic inflammation.

43. The vessel of claim 41, wherein the deleterious sequelae comprise systemic tissue injury.

44. The vessel of claim 41, wherein the carbon monoxide gas is in admixture with an oxygen-containing gas.

45. The vessel of claim 44, wherein the carbon monoxide gas is present in the admixture at a concentration of at least about 0.025%.

46. The vessel of claim 44, wherein the carbon monoxide gas is present in the admixture at a concentration of at least about 0.05%.

47. The vessel of claim 44, wherein the carbon monoxide gas is present in the admixture at a concentration of at least about 0.10%.

48. The vessel of claim 44, wherein the carbon monoxide gas is present in the admixture at a concentration of at least about 1.0%.

49. The vessel of claim 44, wherein the carbon monoxide gas is present in the admixture at a concentration of at least about 2.0%.

50. A vessel comprising whole blood, or a blood component, that is partially or completely saturated with carbon monoxide, the vessel bearing a label indicating that the whole blood or blood component can be administered to a patient to reduce deleterious sequelae of hemorrhagic shock.

51. A business method comprising:
(a) providing whole blood or a blood component suitable for transfusion into a patient;
(b) treating the blood or blood component with carbon monoxide to produce a blood/carbon monoxide product; and
(c) supplying the blood/carbon monoxide product to a customer with instructions to administer the blood/carbon monoxide product to a patient in need of a transfusion.

52. The business method of claim 51, wherein (b) comprises exposing the blood to an atmosphere comprising carbon monoxide.

53. The business method of claim 51, wherein the customer is a hospital or caregiver.

54. The business method of claim 51, wherein the instructions include instructions to administer the blood/carbon monoxide product to a patient who has suffered significant blood loss.
